(19) United States
(12) Patent Application Publication
(10) Pub. No.: US 2025/0090630 A1
(43) Pub. Date: Mar. 20, 2025

(54) USE OF PBX1 IN PROMOTING HAIR GROWTH

(71) Applicant: JILIN UNIVERSITY, Changchun City (CN)

(72) Inventors: Jinyu LIU, Changchun City (CN); Dan LIU, Changchun City (CN)

(21) Appl. No.: 18/962,072

(22) Filed: Nov. 27, 2024

Related U.S. Application Data
(63) Continuation of application No. PCT/CN2023/123666, filed on Oct. 10, 2023.

(30) Foreign Application Priority Data
Jun. 8, 2023 (CN) 202310677117.2

(51) Int. Cl. A61K 38/17; A61K 38/16; C12N 15/86

(52) U.S. Cl. CPC A61K 38/1709; A61K 38/162; C12N 15/86; C12N 2740/15043

(57) ABSTRACT

The present invention provides use of PBX1 or TAT-PBX1 as an active ingredient in promoting hair growth such as use in the preparation of a drug for promoting hair growth and use in the preparation of a medical device for promoting hair growth. It is verified that an umbilical cord mesenchymal stem cell or a culture supernatant loading or over-expressing a TAT-PBX1 fusion protein, or the TAT-PBX1 fusion protein has the ability of significantly promoting hair regeneration.

### TECHNICAL FIELD

The present invention relates to use of biological substances and particularly relates to use of PBX1 in promoting hair growth and use of the PBX1 as an active ingredient in the preparation of a drug or a medical device for promoting hair growth.

### BACKGROUND

The hair follicle is one of the appendages of human and mammalian skin, and originates from the interaction of the epidermis and the mesenchyme during the embryonic development. The hair follicle of human and mammals continuously goes through an anagen phase mainly comprising cell proliferation, a catagen phase mainly comprising cell apoptosis and a telogen phase when cells are in a relative resting state, so as to form a hair follicle cycle. The hair follicle cycle accompanies the life of human and any factor that causes a disturbance in the homeostasis of the hair follicle cycle results in alopecia.

In practice, minoxidil, finasteride and other drugs are mainly used in treating androgenetic alopecia. The minoxidil is originally a drug for treating hypertension. Later researches find that the minoxidil can reasonably control the hair follicle cycle and can be used for treating alopecia. The finasteride administration may disrupt the balance between estrogen and androgen, thereby leading to adverse effects including decreased sexual function. The methods for treating alopecia areata comprise treatment by using glucocorticoid drugs and immunosuppressant drugs, photoelectric treatment and the like. The methods have more adverse reactions, higher cost and high recurrence rate after drug withdrawal.

The researches show that the hair follicle contains various stem cells, including keratinocyte stem cells, melanocyte stem cells, mesenchymal stem cells and neural crest stem cells. The stem cells form an interactive stem cell network in time and space, participate in hair generation, hair growth and hair follicle cycle circulation, cooperate with each other, maintain hair follicle homeostasis together, and enable hairs to regenerate. It is the key for hair regeneration and hair growth to promote the hair follicle to change from the telogen phase to the anagen phase, prolong the anagen phase of the hair follicle and shorten the telogen phase of the hair follicle.

It is known that the common causes of various alopecia types are apoptosis and dysfunction of hair follicle stem cells caused by hormone hypersecretion, immune disorder and inflammatory response. To solve the fundamental problems of the alopecia, the apoptosis of hair follicle stem cells is inhibited, and hair follicle homeostasis and normal operation of the hair follicle cycle are maintained. Although there are reports on the fact that mesenchymal stem cells can promote hair growth, there are still large individual differences in practice. One reason is that the mesenchymal stem cells are only one of the aforementioned four hair follicle stem cells, while the synergistic effect of the four stem cells are required for hair regeneration to have the potential for hair regeneration.

Therefore, the synergistic effect of the four stem cells in regulating and participating hair growth and hair follicle cycle circulation is a key means for treating alopecia.

### SUMMARY

An objective of the present invention is to provide use of PBX1 as an active ingredient in the preparation of a drug for promoting hair growth.

Another objective of the present invention is to provide use of PBX1 as an active ingredient in the preparation of a medical device for promoting hair growth.

PBX1 is a transcription factor for promoting the proliferation of mesenchymal stem cells of hair follicles. However, there is no evidence that whether cells (such as mesenchymal stem cells) over-expressing the PBX1 have the effects of promoting hair growth and whether the effect of promoting hair growth by the PBX1 is better than that by the mesenchymal stem cells.

Cell penetrating peptides (CPPs) have been used to deliver a variety of molecules, particularly large molecular weight active proteins, from the outside of cells to the inside of the cells, such as protamine, TAT and R9, wherein the trans-activator of transcription (TAT) is a peptide derived from human immunodeficiency virus, one of the most commonly used cell penetrating peptides. Many researches have shown that the TAT, when bound to the cells, enhances protein uptake by the cells.

The PBX1 provided by the present invention is present in a culture solution of cells (such as mesenchymal stem cells, neural crest stem cells, melanocyte stem cells and fibroblasts). In order to facilitate transmembrane, the PBX1 generally further comprises a membrane-penetrating peptide such as TAT.

Since a PBX1 protein contained in a cell culture supernatant can enter a cell due to the TAT membrane-penetrating peptide, a cell or a culture supernatant over-expressing TAT-PBX1 provided by the present invention can over-express a TAT-PBX1 protein by infecting the cell by a lentivirus.

A TAT-PBX1 fusion protein provided by the present invention is an exogenous protein expressed by Escherichia coli or a yeast or a CHO cell. The TAT cell membrane-penetrating peptide enhances the protein uptake of the mesenchymal stem cells, such that the PBX1 can penetrate through cell membranes to enter cell nuclei. Besides, the experimental results prove that the TAT-PBX1 obtained by prokaryotic expression still has the transcriptional activity after entering the cell nuclei.

The TAT-PBX1 fusion protein provided by the present invention is packaged by a lentivirus, an adenovirus, an adeno-associated virus or a retrovirus and then is transduced into a cell (such as mesenchymal stem cells, neural crest stem cells, melanocyte stem cells and fibroblasts).

Alopecia including androgen alopecia (AGA), alopecia areata (AA) and telogen effluvium (TE) is essentially apoptosis and dysfunction of hair follicle stem cells caused by hormone hypersecretion, immune disorder and inflammatory response. The PBX1 and the TAT-PBX1 of the present invention increase the number of hair follicles in dermal tissues, the diameters of hair bulbs, the lengths of hair shafts, and can maintain hair follicle homeostasis and promote the hair follicles to rapidly enter an anagen phase, thereby promoting hair growth and solving an essential problem of alopecia. The PBX1 and the TAT-PBX1 are suitable for all the diseases caused by the alopecia. A drug for promoting hair growth prepared by taking the PBX1 or the TAT-PBX1 as an active ingredient, or a medical devices (such as a microneedles) loading the active ingredient are used for treating diseases such as AGA, AA and TE.

The PBX1 or the TAT-PBX1 prepared by the present invention is used as an active ingredient for promoting hair growth. The PBX1 or the TAT-PBX1 is mixed with other adjuvants to prepare a drug (preparation) for promoting hair growth, which can be used for preventing alopecia, caring hair and promoting hair growth.

The pharmaceutical adjuvants can be those conventionally used in various preparations, such as but not limited to isotonic agents, buffers, flavoring agents, excipients, fillers, binders, disintegrants, lubricants and the like; and may be selected for use in accordance with substances, such as emulsifiers, solubilizers, antimicrobial agents, analgesics, and antioxidants. The adjuvants can effectively improve the stability and solubility of compounds contained in a composition or change the release rate, absorption rate and the like of the compounds, thereby improving the metabolism of various compounds in organisms and further enhancing the administration effect of the composition.

The adjuvants used in an aqueous solution injection generally include isotonic agents and buffers, as well as necessary emulsifiers (such as Tweeen-80, Pluronic, Poloxamer, etc.), solutizers and antimicrobial agents. In addition, other pharmaceutically acceptable pharmaceutical adjuvants such as antioxidants, pH regulators, analgesics and the like are further comprised.

The adjuvants used in the preparation of a liquid preparation generally include solvents, water, oils (such as fatty acids), emulsifiers, necessary preservatives and the like.

The various adjuvants and a culture medium supernatant of the present invention are prepared into a dosage form which is beneficial to administration, such as but not limited to aqueous solution injections, powder injections, powders, patches, suppositories, emulsions, creams, gels, aerosols, sprays, powder sprays, sustained release agents and controlled release agents. In addition, the adjuvants can further be used to realize specific administration purposes or modes such as sustained release administration, controlled release administration, pulse administration and the like. The adjuvants are such as but not limited to gelatin, albumin, chitosan, polyether and polyester-based high polymer materials such as but not limited to polyethylene glycol, polyurethane, polycarbonate, copolymers thereof and the like. The main indications of the “beneficial to administration” are but not limited to improving the therapeutic effect, improving the bioavailability, reducing the toxic and side effects, improving the patient compliance and the like.

Medicated medical devices prepared by combining drugs with medical devices have also become common such as dressings containing PBX1 or TAT-PBX1 of the present invention. The PBX1 or the TAT-PBX1 of the present invention is further loaded or coated onto a material as an active ingredient for preparing a medical device for promoting hair growth. Common scaffold materials are as follows: PLA, PLGA, GelMA, metals and the like. Besides, the PBX1 or the TAT-PBX1 is mixed with a biocompatible degradable material to prepare a microneedle and a microneedle array, or to be loaded in a metal microneedle to be prepared into a microneedle chip. After the microneedle penetrates the skin, the PBX1 or the TAT-PBX1 is released into an epithelial tissue so as to regulate a hair follicle cycle and promote hair growth.

The stem cells (such as mesenchymal stem cells) are also directly used as a vector of the PBX1 for promoting hair growth. The stem cells over-expressing the PBX1 or the TAT-PBX1 promote hair growth, and are used in preventing alopecia, caring hair and promoting hair growth, and treating diseases such as AGA, AA and TE.

### BRIEF DESCRIPTION OF THE DRAWINGS

FIG. 1 is a map of a TAT-PBX1 plasmid;

FIG. 2 shows the results of light field observation of a control group and stem cells over-expressing TAT-PBX1;

FIG. 3 is Western blot of the control group and over-expressed TAT-PBX1;

FIG. 4 is Western blot showing that a TAT-PBX1 protein is contained in a culture supernatant of umbilical cord mesenchymal stem cells over-expressing TAT-PBX1;

FIG. 5 is a general perspective view showing that the culture supernatant of the stem cells over-expressing TAT-PBX1 significantly promotes hair regeneration of mice;

FIG. 6 shows the results of HE staining of hair follicles of mice in the control group and a TAT-PBX1-overexpressing stem cell culture supernatant group;

FIG. 7 shows the results of the number of hair follicles of the mice in the control group and the TAT-PBX1-overexpressing stem cell culture supernatant group;

FIG. 8 shows the results of the diameters of hair bulbs of the mice in the control group and the TAT-PBX1-overexpressing stem cell culture supernatant group;

FIG. 9 is a general perspective view showing that a TAT-PBX1 fusion protein significantly promotes hair regeneration of mice;

FIG. 10 shows the results of HE staining of hair follicles of the mice in the control group and a TAT-PBX1 fusion protein group;

FIG. 11 shows the results of the number of hair follicles per visual field of the mice in the control group and the TAT-PBX1 fusion protein group; and

FIG. 12 shows the results of the lengths of hair shafts of the mice in the control group and the TAT-PBX1 fusion protein group.

### DETAILED DESCRIPTION

The technical solutions of the present invention are described below with reference to the accompanying drawings. The examples of the present invention are only intended to explain, rather than to limit the technical solutions of the present invention. Although the present invention is described in detail with reference to the preferred examples, a person skilled in the art should understand that modifications or equivalent substitutions may be made to the technical solutions of the present invention without departing from the spirit and scope of the technical solutions of the present invention, and such modifications or equivalent substitutions should be included within the scope of the claims of the present invention.

Various test methods used in the following examples of the present invention are specified as follows:

1) Preparation of TAT-PBX1 fusion protein

Preparation of TAT-PBX1 fusion protein: a gene encoding PBX1 is amplified by PCR, the PCR product is ligated to a cloning vector pT-EASY by T-A cloning, and white clones which are correctly ligated are then picked by blue-white screening. After bacterial liquid PCR, enzyme digestion identification and gene sequencing identification, a PBX1-encoding gene fragment in the positive clones is inserted into a prokaryotic expression vector pTAT-HA (purchased from Addgene plasmid #35612) through enzyme digestion and T4 connection, then transferred into a prokaryotic expression strain Rosetta (DE3) (purchased from Tiangen Biotech Co., Ltd.), and subjected to screening through ampicillin resistance, so as to obtain a positive transformant. The positive transformant was activated overnight at 37° C. in an expression culture medium, subjected to amplification culture, and centrifuged to collect a precipitate; and the precipitate was placed in a lysate for ultrasonic disruption and dissolution, and centrifuged for 12,000 g×30 min, and a supernatant was collected. After the supernatant was filtered by using a 0.45-μm filter, the supernatant was purified by using a nickel column affinity chromatography, and renatured by using a pulse dilution mode. The renaturated solution was dialyzed and ultrafiltered to obtain a high-concentration TAT-PBX1 fusion protein.

The purification of the TAT-PBX1 fusion protein comprises the following steps: a Ni²⁺-NTA-agarose column is used and equilibrated with a binding buffer (1-200 mM of Tris-HCl, 1-200 mM of imidazole, 1-500 mM of NaCl and 1-8 M of urea at the pH of 9.5), the bacterial supernatant is loaded onto the column, the chromatographic column is rinsed with a washing buffer (1-200 mM of Tris-HCl, 1-509 mM of imidazole, 500 mM of NaCl and 8 M of urea at the pH of 9.5), and the TAT-PBX1 fusion protein bound to the chromatographic column is eluted with an elution buffer (1-100 mM of Tris-HCl, 1-500 mM of imidazole, 1-500 mM of sodium chloride and 1-8 M of urea at the pH of 9.5).

The renaturation of the TAT-PBX1 fusion protein comprises the following steps: the eluted sample is renaturated in a renaturation buffer containing 1-20 mM of Tris, 1-20 mM of glycine, 1-10 mM of EDTA and 0.1-0.5 M of arginine at the pH of 9.5 according to the dilution ratio of 1:(1-20). After the renaturation is completed, the supernatant is introduced into a dialysis bag and dialyzed overnight at 4° C. using a 1-10 mM phosphate buffer (pH of 5.4). After this, the denatured protein is concentrated by using a 30-kDa ultrafiltration tube (purchased from Millipore, USA). The concentration of the fusion protein is determined by using a BCA protein detection kit.

2) Preparation of umbilical cord mesenchymal stem cells over-expressing TAT-PBX1

An empty vector TAT is packaged as a lentiviral particle: when the density of 293T cells (given by Institute of Biochemistry and Cell Biology, Shanghai Institute of Life Sciences, Chinese Academy of Sciences) reaches 70%-75%, transfection is performed, namely, firstly, 500 μL of a serum-free culture medium (Opti-MEM), 1-20 μg of a TAT plasmid (constructed in the laboratory), 3-15 μg of a packaging plasmid PAX₂ (purchased from Addgene) and 1-5 μg of PMD2G (purchased from Addgene) are added into a 1.5-mL EP tube; and at the same time, 1-50 μL of a LipoFiter™ transfection reagent (Thermo Fisher) is added to 500 μL of Opti-MEM, and both are incubated at room temperature for 5-100 min. The plasmid-containing Opti-MEM is added dropwise to the Opti-MEM containing lipofectamine 2000 (Thermo Fisher), incubated at room temperature for 15-20 min, and added dropwise to the 293T. The cells were cultured at 37° C. and 5% CO₂. After the cells are cultured for 6-8 h, DMEM containing 10% FBS is changed. After the cells are continuously cultured for 36 h, a DMEM supernatant containing virus particles is collected and centrifuged at 3,000 r/min for 5-10 min. The supernatant is collected and filtered through a 0.45-μm filter for use.

TAT-PBX1 is packaged as a lentiviral particle: when the cell density of 293T reaches 70%-75%, transfection is performed, namely, firstly, 500 μL of a serum-free culture medium (Opti-MEM), 1-30 μg of a TAT-PBX1 plasmid (constructed in the laboratory), 3-10 μg of a packaging plasmid PAX₂ (purchased from Addgene) and 1-5 μg of PMD2G (purchased from Addgene) are added into a 1.5-mL EP tube; and at the same time, 1-30 μL of a lipofectamine 2000 is added to 500 μL of Opti-MEM, and both are incubated at room temperature for 5-10 min. The Opti-MEM containing the plasmid and the Opti-MEM containing the lipofectamine 2000 are incubated at room temperature for 15-20 min, added dropwise to the 293T (given by Institute of Biochemistry and Cell Biology, Shanghai Institute of Life Sciences, Chinese Academy of Sciences), cultured at 37° C. and 5% CO₂ for 6-8 h, and the culture medium is replaced with DMEM containing 10% FBS. The cells are continuously cultured for 36 h, and the supernatant of the DMEM containing virus particles is collected and centrifuged at 3,000 r/min for 5-10 min. The supernatant is collected and filtered through a 0.45-μm filter for use.

3) Preparation of umbilical cord mesenchymal stem cells loading TAT-PBX1 protein

Umbilical cord mesenchymal stem cells are cultured by using DMEM. When the cell density reaches 70%-75%, a TAT-PBX1 fusion protein is added into the culture medium to the final concentration of 1 ng/ml-100 μg/ml. After the cells are continuously cultured for 1 min to 120 h, the TAT-PBX1 fusion protein penetrates through cell membranes to enter cell nuclei so as to promote the cell growth.

The umbilical cord mesenchymal stem cells are cultured by using DMEM. When the cell density reaches 70%-75%, the culture medium for the umbilical cord mesenchymal stem cells is discarded, and a mixed solution of 7.5 μL of polybrene, 7.5 μL of bFGF and a virus supernatant is added. After 24 h of transduction, the umbilical cord mesenchymal stem cells over-expressing TAT-PBX1 are successfully established, and the TAT-PBX1 protein penetrates through the cell membranes and is secreted into a cell culture supernatant.

4) Preparation of umbilical cord mesenchymal stem cell culture supernatant over-expressing or loading TAT-PBX1 protein

The umbilical cord mesenchymal stem cells over-expressing or loading the TAT-PBX1 protein are cultured by using the DMEM. When the cell density reaches 70%-75%, the culture medium is replaced with DMEM free of phenol red and serum. After the cells are continuously cultured for 24 h, a cell culture supernatant is collected and the protein concentration is quantitatively determined by using BCA. The culture supernatant is freeze-dried in a vacuum freezer, scaled and stored at −80° C. for use.

5) TAT-PBX1 packaged by adenovirus

When 293A cells (Wuhan Pricella Biotechnology Co., Ltd.) reach 50%-70% of confluency, transfection is performed, TAT-PBX1 is recombined into an AdMAX system (HANBIO). The system consists of double vectors of a pHBAd series shuttle plasmid and an adenovirus skeleton vector pBHGlox(delta)E13Cre (HANBIO). After 6 h of the transfection, the culture medium is replaced with a fresh culture solution. Before virus collection, whether virus plaques are formed is observed. To limit the spread of the viruses and enable the formation of plaques to be better, agarose at a low melting point is generally added to the culture solution and small plaques can be seen under a microscope, generally on day 10 to day 21 after the transfection. After the plaque formation, the plaques are picked up with the agarose and placed in a 1-ml fresh culture medium overnight. Generally, 3-6 plaques are picked, then the titers are compared, and the one plague with the highest titer is used for a subsequent experiment. On the next day, the viruses in the culture medium are added to a fresh 293A cell culture solution for small-amount expansion of the viruses. When the cells have plaques again, the cells and the supernatant are collected, and repeatedly freeze-thawed for three times to collect the viruses, the viruses are taken as P1-generation viruses, 293A cells are infected with the P1-generation adenoviruses, third generation infection is performed continuously, massive expansion of the adenoviruses is performed to the P4 generation, and the viruses are collected after the plaques are formed, and purified and concentrated in vitro. The umbilical cord mesenchymal stem cells are cultured by using DMEM. When the cell density reaches 70%-75%, 5 μl of the adenoviruses is added into each 1 ml of the culture medium. After 72 h of the transduction, TAT-PBX1 umbilical cord mesenchymal stem cells which are successfully transduced are obtained by puromycin screening and verification is performed by using Western blot.

6) TAT-PBX1 packaged by adeno-associated virus

When the 293A cells reach 50%-70% of confluency, transfection is performed and the culture medium is replaced with a serum-free culture medium within 2 h before the transfection. Opti MEM and a LipoFiter™ transfection reagent (Thermo Fisher) are preheated in a water bath at 37° C. and returned to room temperature for use. Before use, both are needed to be shaken evenly. TAT-PBX1 is recombined into a pAAV-IRES-PURO vector (Addgene) to construct a TAT-PBX1 plasmid of an AAV vector, the plasmid is transfected into the 293A cells, after 6 h, the culture medium is replaced with a fresh complete culture medium, after 72 h, the cells containing the AAV particles are gently scraped, collected in a 15-mL centrifuge tube and centrifuged at 150 rpm/min for 3 min. After the supernatant is removed, a cell precipitate is reserved. The cells are resuspended with 300 μL of LPBS after the cell precipitate is washed once with PBS. The centrifuge tube containing the cell suspension is repeatedly freeze-thawed 3 times in liquid nitrogen and 37° C. water bath. The cell suspension is centrifuged at 3,000 rpm/min for 5 min at 4° C., a lysate supernatant containing the AAV particles is filtered through a 0.45-μM filter and collected. To remove residual plasmid DNA and cellular genome in a crude viral extract, 0.1. μL of a Benonase enzyme (Sigma) is added to 1 mL of a viral solution and subjected to a water bath at 37° C. for 1 h. Then the mixture is centrifuged at 600 rpm/min for 10 min at 4° C. to obtain a supernatant. Subsequently, the viruses are purified and concentrated. The umbilical cord mesenchymal stem cells are cultured by using DMEM. When the cell density reaches 70%-75%, 0.5 ml of the adeno-associated viruses is added into each 1 ml of the culture medium. After 72 h of the transduction, TAT-PBX1 umbilical cord mesenchymal stem cells which are successfully transduced are obtained by puromycin screening and verification is performed by using Western blot.

7) Test for promoting hair growth

7-week-old SPF-grade male C57BL/6 mice are randomly divided into 5 groups, namely, a blank control group, a solvent control group, an umbilical cord mesenchymal stem cell culture supernatant group, an empty vector umbilical cord mesenchymal stem cell culture supernatant group and a TAT-PBX1 over-expressing umbilical cord mesenchymal stem cell culture supernatant group. The mice are fed at constant temperature of 22° C. and constant humidity, and in the quiet environment. The hairs of the dorsal skin of the mice are removed and depilated on the dorsal side of the mice using a melted paraffin/rosin mixture (1:1) to expose a skin of approximately 4 cm×2 cm size to ensure that all hair follicles in the dorsal skin of the depilated mice are in the same hair follicle cycle.

First, a freeze-dried powder of an umbilical cord mesenchymal stem cell culture supernatant over-expressing TAT-PBX1 or a freeze-dried powder of an umbilical cord mesenchymal stem cell supernatant loading a TAT-PBX1 protein is dissolved, and then a solvent, an umbilical cord mesenchymal stem cell culture supernatant, an empty vector umbilical cord mesenchymal stem cell culture supernatant and the umbilical cord mesenchymal stem cell culture supernatant over-expressing TAT-PBX1 are properly diluted and subcutaneously injected into the depilated part of the back of the mice. The mice are injected twice. After 14 days, the hair growth of the mice is observed and photographed.

The mice are sacrificed by cervical dislocation and the hairs on the back are shaved off with electric clippers and shavers. The lower skin tissue of the depilated part of the mice is taken, the subcutaneous fat layer is removed, and the rest is prepared into paraffin sections. After HE staining, the growth of the hair follicles is observed under a microscope.

8) Statistical analysis

All data are expressed as means+standard deviation. Statistical analysis and statistical chart generation are both performed using a GraphPad Prism8 software. For comparison among groups, a one-way ANOVA test is used if the data are normally distributed and a least significant difference test (LSI D) is used for multiple comparisons among groups; or a Kruskal-Wallis test is used if the data are not unequal in variance with a test level a-0.05. P<0.05 is statistically significant and P<0.01 is statistically significantly different.

Example 1 Preparation and verification of stem cells over-expressing TAT-PBX1

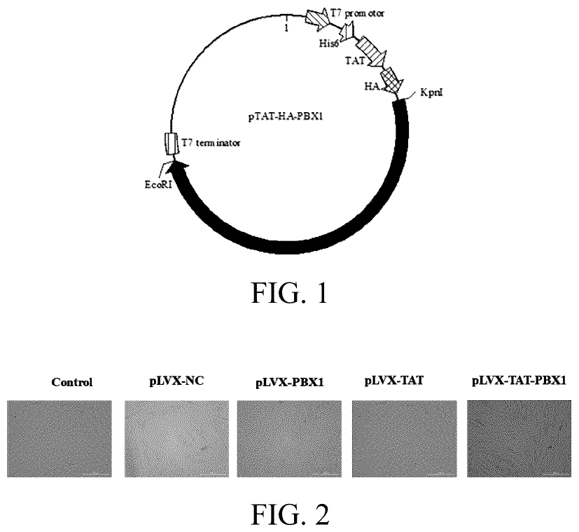

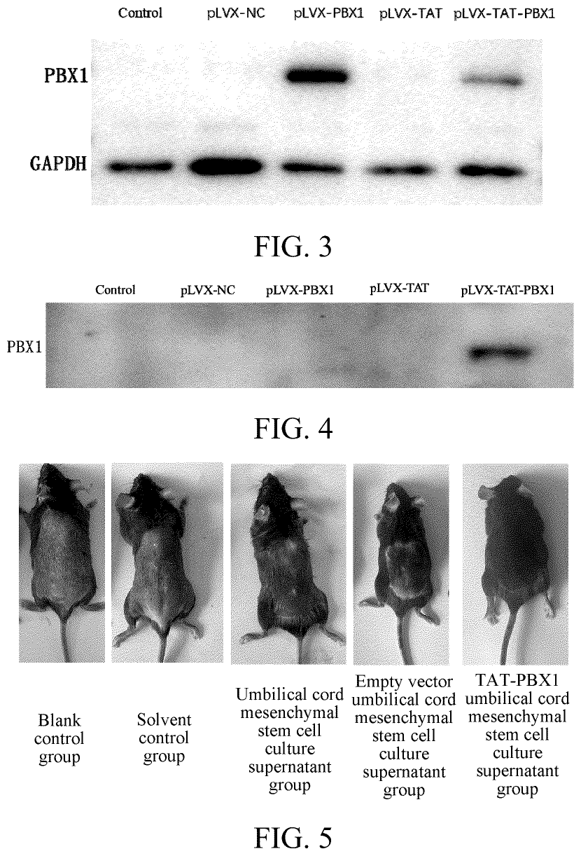

The umbilical cord mesenchymal stem cells were cultured by using the DMEM. When the cells reached the density of 70%-75%, subculture was performed according to the proportion of 1:3. Subsequently, lentivirus (packaged in this laboratory) infection was performed. The patent took the lentivirus as a vector for introducing a TAT-PBX1 target plasmid as an example: blank control, empty vector (GENE), PBX1 (GENE), TAT (purchased from Addgene) and TAT-PBX1 (constructed by this laboratory) (FIG. 1). Packaging plasmids: psPAX2-gag/pol/tat/rev and pMD2.G-VSVG (purchased from Addgene). After the PBX1 or the TAT-PBX1 was packaged as a lentiviral particle in 293T cells (given by Institute of Biochemistry and Cell Biology, Shanghai Institute of Life Sciences, Chinese Academy of Sciences), the cells were transduced into the umbilical cord mesenchymal stem cells. After the transduction for 72 h, the PBX1 transduction was observed by a fluorescence microscopy. The TAT-PBX1 over-expressing umbilical cord mesenchymal stem cells were obtained by puromycin screening (FIG. 2) and verification was performed by using Western blot (FIG. 3).

Example 2 Preparation and verification of umbilical cord mesenchymal stem cell culture supernatant loading TAT-PBX1 protein

The umbilical cord mesenchymal stem cells loading the TAT-PBX1 protein were cultured by using the DMEM. When the cell density reached 70%-75%, the culture medium was replaced with DMEM free of phenol red and serum. After the cells are continuously cultured for 24 h, a cell culture supernatant is collected and the protein concentration is quantitatively determined by using BCA. The culture supernatant was freeze-dried in a vacuum freezer, sealed and stored at −80° C. for use. The expression of the TAT-PBX1 protein in the cell culture supernatant was detected by using Western blot (FIG. 4).

Example 3 Verification of umbilical cord mesenchymal stem cell culture supernatant over-expressing TAT-PBX1 in promoting hair growth

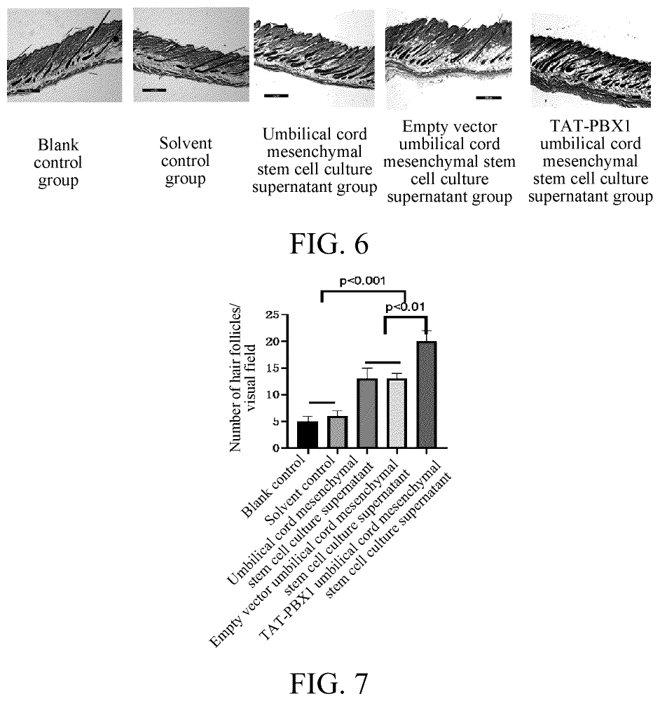

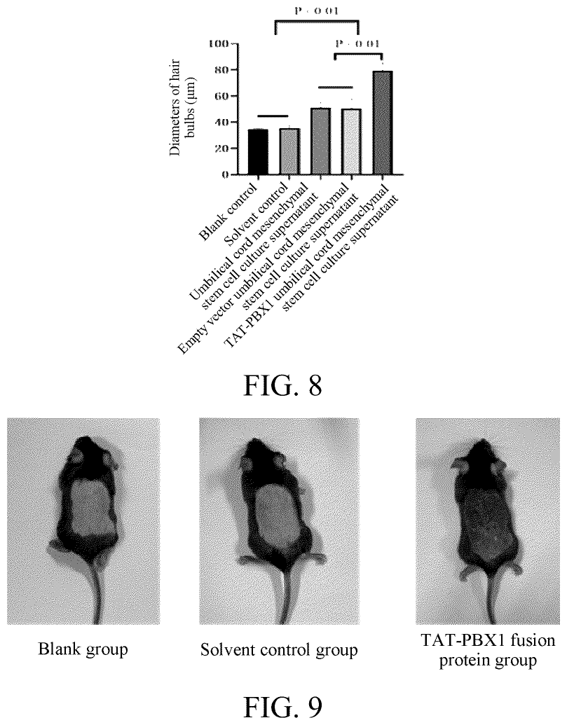

Compared with the solvent group, the umbilical cord mesenchymal stem cell culture supernatant, the empty vector umbilical cord mesenchymal stem cell culture supernatant and the umbilical cord mesenchymal stem cell culture supernatant over-expressing TAT-PBX1 all significantly promoted the hair regeneration of mice. Moreover, compared with the umbilical cord mesenchymal stem cell culture supernatant and the empty vector umbilical cord mesenchymal stem cell culture supernatant, the umbilical cord mesenchymal stem cell culture supernatant over-expressing TAT-PBX1 can more significantly promote the hair regeneration of the mice (FIG. 5). HE staining showed the hair follicles of the mice in the solvent group were still in a telogen phase, but the hair follicles in the skin of the mice in the TAT-PBX1 over-expressing umbilical cord mesenchymal stem cell culture supernatant group entered an anagen phase (FIG. 6). Compared with the solvent group, the number of the hair follicles in the skin (FIG. 7) and the diameters of hair bulbs (FIG. 8) of the mice in the TAT-PBX1 over-expressing umbilical cord mesenchymal stem cell culture supernatant group were obviously increased.

Example 4 Verification of TAT-PBX1 fusion protein in promoting hair growth

Compared with the control group, the hairs of the mice subcutaneously injected with the TAT-PBX1 fusion protein entered the anagen phase more rapidly (FIG. 9).

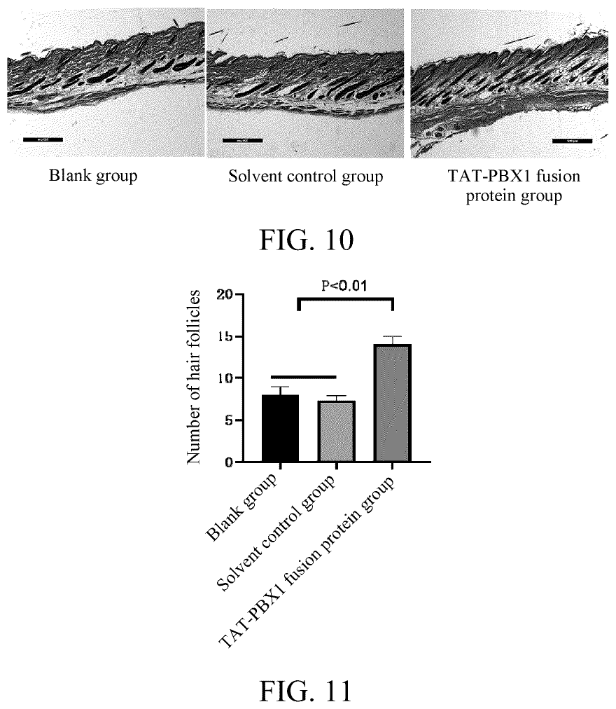

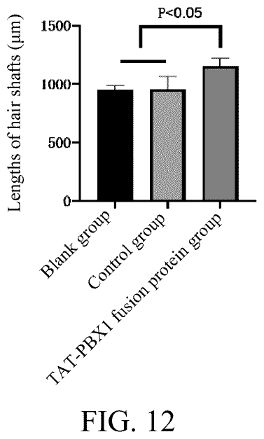

The analysis of the HE staining results (see FIG. 10) showed that compared with the solvent group, the mice in the TAT-PBX1 group had increased number of hair follicles per unit area of skin (FIG. 11) and increased lengths of hair shafts (FIG. 12). The results showed that the hair follicles in the skin of the mice in the TAT-PBX1 fusion protein group rapidly entered the anagen phase from the telogen phase, such that the number of the hair follicles and the lengths of the hair shafts of the mice were increased.

Thus, it can be seen that the umbilical cord mesenchymal stem cell culture supernatant over-expressing TAT-PBX1 or the TAT-PBX1 fusion protein had the ability of significantly promoting hair regeneration.

What is claimed is:

1. Use of PBX1 or TAT-PBX1 as an active ingredient in the preparation of a drug or a medical device for promoting hair growth.

2. The use according to claim 1, wherein the medical device is a microneedle or a microneedle array.

3. The use according to claim 1, wherein the PBX1 or the TAT-PBX1 is packaged by a lentivirus, an adenovirus, an adeno-associated virus or a retrovirus and then is transduced into a cell.

4. The use according to claim 1, wherein the PBX1 or the TAT-PBX1 is contained in a cell or is secreted outside the cell after being expressed by the cell.

5. The use according to claim 4, wherein the cell is a mesenchymal stem cell, a neural crest stem cell, a melanocyte stem cell and a fibroblast.

6. The use according to claim 4, wherein the cell is a vector and the PBX1 or the TAT-PBX1 is loaded in the cell.

7. The use according to claim 4, wherein the cell over-expresses the PBX1 or the TAT-PBX1.

8. The use according to claim 4 in promoting a hair follicle cycle.

9. Use of a fusion protein in the preparation of a drug for promoting hair growth, wherein the fusion protein is a TAT-PBX1 fusion protein.

10. The use according to claim 9, wherein the fusion protein is expressed by Escherichia coli or a yeast or a CHO cell.
